(19) United States
(12) Patent Application Publication
(10) Pub. No.: US 2005/0074443 A1
(43) Pub. Date: Apr. 7, 2005

(54) Methods of attenuating autoimmune disease and compositions useful therefor

(76) Inventor: Benjamin V. Treadwell, Marco Island, FL (US)

Correspondence Address: KIRKPATRICK & LOCKHART NICHOLSON GRAHAM LLP, 535 SMITHFIELD STREET, PITTSBURGH, PA, 15222, US

(21) Appl. No.: 10/678,371

(22) Filed: Oct. 3, 2003

(51) Int. Cl. A61K 38/43; A61K 31/714; A61K 31/202; A61K 31/045; A61K 31/20; A61K 31/59

(52) U.S. Cl. 424/94.1; 514/167; 514/52; 514/560; 514/724

(57) ABSTRACT

The present invention relates to the reduction or attenuation a specific composition has on immunological tissue-destructive conditions associated with specific autoimmune diseases. The composition includes a long chain primary aliphatic alcohol, and one or more of three cofactors: a D Vitamin, a B₁₂ Vitamin, a coenzyme Q, and an omega-3 fatty acid. The composition functions coordinately to modify multiple autoimmune disease risk factors and symptoms associated with the autoimmune disease. The compounds work in a synergistic manner to attenuate tissue-destructive inflammation.

### STATEMENT REGARDING FEDERAL SUPPORT

Not Applicable

### CROSS REFERENCE TO RELATED APPLICATIONS

Not Applicable

### BACKGROUND

1. Field of the Invention

Long-chain aliphatic alcohols, omega-3 fatty acids, Coenzyme Q10 and vitamin compositions for attenuation of pathologies as well as repair of damaged tissues associated with autoimmune diseases.

2. Description of the Related Art

Recent research has demonstrated a number of inflammatory elements associated with certain autoimmune diseases. Such inflammatory elements, when present in the tissues of the body in elevated amounts, create a condition that promotes tissue destruction. Some of the diseases involve the nervous system and include, multiple sclerosis (MS) others involve the tissues within or surrounding articulating joint surfaces and include rheumatoid arthritis.

Multiple sclerosis, known as “The Great Crippler of Young Adults,” is a chronic disabling disease of the central nervous system. Multiple sclerosis usually appears between the ages of 20 and 40. Multiple sclerosis is now considered to be an autoimmune disease because of the heightened action of white blood cells that can attack the myelin of the central nervous system. The myelin is a fatty sheath that surrounds, insulates, and protects the nerve fibers. Myelin damage causes nerve signals to be slowed, shorted, or blocked, creating some of the classic symptoms of multiple sclerosis. Anti-myelin antibodies have been demonstrated to be present in the serum of patients with multiple sclerosis, supporting the hypothesis that multiple sclerosis is an autoimmune disease. Furthermore, because of the definitive nature of antibodies to two specific myelin proteins, myelin oligodendrocyte glycoprotein (MOG), and myelin basic protein (MBP), present in the serum of multiple sclerosis patients, an assay has been developed for diagnostic purposes, which is based on the detecting the presence of one or both of these antibodies.

Autoimmune diseases, such as multiple sclerosis and rheumatoid arthritis, are part of a larger group of autoimmune diseases that affect over 8.5 million people in the United States. Of these, a disproportionate number are women. For instance, in multiple sclerosis and rheumatoid arthritis, the incidence is between 2 and 3 women to every man. Rheumatoid arthritis affects over 4 million and multiple sclerosis affects about 500,000 people in the United States, alone. Both diseases are debilitating and often result in complete immobilization. Other autoimmune diseases, whether organ-specific, such as Grave's disease and Insulin-Dependent Diabetes Mellitus, or systemic, such as Systemic Lupus Erythematosis and Scleroderma, affect significant populations.

Patients with autoimmune diseases have significant abnormalities of immunoregulatory factors. The abnormality stems from an imbalance in the relative levels of factors involved in immune regulation, as a consequence of self-directed immunity. The resulting inflammation leads to the destruction of tissues surrounding the area under attack, such as the myelin sheath surrounding nervous tissue with multiple sclerosis patients, and in the case of rheumatoid arthritis, the cartilage covering the articulating surface of a joint as well as surrounding joint tissue.

Exacerbation of autoimmune disease is associated with elevated serum levels of cellular factors that promote inflammation, including tumor necrosis factor alpha (TNF-α) and interferon gamma (IFN-γ). It appears that an imbalance in the ratio of two additional interleukins, IL-12 and IL-10 in these patients may trigger the production of TNF-α and IFN-γ. These factors, IL-12 and IL-10, are produced by thymus-derived immune cells called helper T1 (T_{H}1) and helper T2 (T_{H}2) cells respectively. Another risk factor, Rho, is a GTP binding protein, and recent information indicates that attenuation of tissue levels of this protein, as well as related proteins, stimulates the synthesis of myelin and neurite growth. Furthermore, Rho also is involved in promoting the migration of inflammatory white blood cells from the vasculature, into the central nervous system. This breech of the blood-brain barrier by inflammatory cells is believed to be a key-step leading to destruction of nerve tissue such as the myelin sheath. Substances that inhibit Rho synthesis or its activity can help prevent nervous tissue destruction.

Rho GTPase activity recently has been demonstrated to specifically facilitate, the transport of activated T cells across the CNS vasculature to enter the neural parenchyma. The mechanism of Rho-promoted transport of inflammatory cells across the vasculature, as well as neurite growth probably involves Rho's effects on the actin cytoskeleton of cells. Rho cycles between the inactive, GDP-bound form and the active, GTP-bound form. When Rho is in the GTP-bound from it inhibits neurite growth by promoting growth cone collapse of the budding neuron. Activated Rho (Rho-GTP) is then acted on by downstream effector proteins, which bind specifically to the activated Rho family GTPases.

Therefore, in patients with multiple sclerosis, where there are numerous antigen activated lymphocytes (anti-MOG and anti-MBP), inhibition of Rho activity could prevent or attenuate the movement of inflammatory cells into the nervous system and thus prevent inflammation and tissue destruction. It appears that the inflammatory lymphocytes bind to a protein, lymphocyte function-associated antigen-1 (LFA-1), which in turn binds to an intercellular adhesion molecule (ICAM) present on the endothelial cells. The bound inflammatory cell (as part of a quarternary complex; lymphocyte-LFA-1-ICAM-endothelial cell) is positioned to traverse the wall of the vessel to enter the parenchyma of the brain. However, this process, the transport of the lymphocyte bound to the endothelial cell lining the wall of the vessel, requires the hydrolysis of GTP (source of energy) to complete the process. The GTP binding protein, Rho, participates in the GTP hydrolytic step, and therefore is required for movement of the lymphocyte into the brain compartment.

Interventional prospective clinical trials, as well as similar trials with animal models, utilize various agents to reduce or modulate the patient's immune response. These treatments have shown some promise in attenuating (but not eliminating) the symptoms of the disease. The treatments appear to function by lowering serum levels of the immune up-regulating factors TNF-α and IFN-γ. All of the agents currently used for treating multiple sclerosis and rheumatoid arthritis have significant side effects, and require injections. One agent is corticosteroids, which attenuate inflammatory disease but have significant toxic effects with long-term use. Another agent useful in treating multiple sclerosis is beta interferon (IFN-β), a cytokine with anti-inflammatory activity. IFN-β commonly is used for multiple sclerosis patients with low to moderate success but also has substantial side effects. IFN-βneeds to be injected and is costly, as are other agents used to treat multiple sclerosis, mitoxantrone, and glatiramer acetate. Mitoxantrone kills leukocytes (inflammatory cells), and therefore has significant toxic, effects. Glatiramer compounds are synthetic peptides with the composition mimicking portions of the myelin protein. The basis for its action is to react with anti-myelin antibodies, thus preventing them from reacting with the patient's myelin sheath. More recently there is evidence from preliminary clinical trials demonstrating encouraging results with statins on attenuating the physical symptoms of relapsing-remitting multiple sclerosis. These drugs also have side effects including, hepatotoxicity, and a more serious disorder, rhabdomyolysis, which can cause death of the patient as well as polyneuropathy.

Many of the wide-ranging health benefits conferred by statin therapy are mediated, not only through their inhibitory effect on cholesterol synthesis, but rather by inhibition of isoprenylation reactions essential to the activation of Rho family GTPases. That inhibition may be the mechanism primarily responsible for the favorable impact of statins on the risk for ischemic stroke, senile dementia, and fractures, as well as the anti-hypertensive and platelet-stabilizing actions of these drugs. The extent of these benefits might suggest that most adults would be wise to take statins. However, owing to the significant expense of statin therapy, as well as the potential for dangerous side effects that mandates regular physician follow-up, this strategy appears to be impractical.

Policosanol, a mixture of long-chain aliphatic alcohols prepared from sugar cane wax, has shown cholesterol-lowering potency comparable to that of statins, and yet appears to be devoid of toxic risk. Recent evidence indicates that policosanol down-regulates cellular expression of HMG-CoA reductase, and thus has the potential to suppress isoprenylation reactions much like statins do. Consistent with this possibility, the results of certain clinical and animal studies demonstrate that policosanol has many effects analogous to those of statins that are not likely explained by reductions of LDL cholesterol. However, unlike statins, policosanol does not directly inhibit HMG-CoA reductase, and even in high concentrations it fails to down-regulate this enzyme by more than 50%—thus likely accounting for the safety of this nutraceutical.

The precise mode of action of policosanol in promoting positive effects on cardiovascular risk factors, such as elevated LDL, lower HDL, elevated total cholesterol level, and elevated triglycerides, currently is not understood. Unlike the statin drugs, the long chain aliphatic alcohols do not appear to inhibit the committed step in cholesterol synthesis, 3-hydroxy-3-methylglutaryl CoA reductase activity. Nevertheless, the effect of both the statin drugs and the long chain aliphatic alcohols is to reduce cholesterol synthesis.

High molecular weight aliphatic alcohols have been used for the treatment of hypercholesterolemia and are disclosed in U.S. Pat. Nos. 5,856,316 and 5,663,156, which describe a process for preparation from sugar cane of high molecular weight primary aliphatic alcohols of about 24 to 34 carbons of a particular quantitative combination. Sorkin, in U.S. Pat. Nos. 5,952,393 and 6,197,832, discloses a composition comprising phytosterol and policosanol and methods of use thereof for reducing serum cholesterol in humans and animals. Perez, in U.S. Pat. No. 6,225,354, describes a mixture of higher molecular weight aliphatic alcohols naturally obtained from beeswax that contain about 24 to 34 carbon atoms. Mixtures of aliphatic alcohols of about 20 to 40 carbons in length are found in natural sources and have demonstrated the ability to lower serum total cholesterol as well as LDL cholesterol. Policosanol is a mixture of high molecular weight aliphatic alcohols, generally ranging from about 24 to 34 carbons in length. These long-chain alcohols can be prepared from rice bran, sugar cane wax, or beeswax. The profile of the aliphatic alcohols differs somewhat depending upon source and method of extraction. However, it is believed that the serum cholesterol-lowering effect is attributable primarily to octacosanol, triacontanol, and dotriacontanol content of the extract.

High molecular weight aliphatic alcohols function by inhibiting the synthesis of cholesterol in the liver and increasing the hepatic reabsorption of LDL, U.S. patent application No. 20030054978. The mechanism involved in this inhibition is not clearly defined, but is believed to occur at the transcriptional or translational level in the expression of HMG-CoA, rather than direct inhibition of HMG-CoA, as is the mechanism with the statins. However, the net result is the same, inhibition of mevalonate synthesis and the subsequent mevalonate-derived molecules, isoprene, cholesterol, and inhibition of the activity of GTP binding proteins, Rho, Rac and Ras.

Double-blind control studies, involving a total of almost 1500 individuals and ranging in length from 6 weeks to 12 months, have found high molecular weight aliphatic alcohols effective for improving cholesterol levels. The results suggest that treatment with as little as about 10 mg high molecular weight aliphatic alcohols per day can reduce LDL cholesterol by about 20 percent or more and total cholesterol by about 15 percent. Some studies found improvement in triglyceride and HDL cholesterol, but others did not. Interestingly, most of these studies enrolled only individuals whose cholesterol levels had not improved with diet alone.

Typical clinical doses of high molecular weight aliphatic alcohols used to lower the elevated serum cholesterol range from about 5 to 10 mg administered twice daily. Several weeks, e.g. two months, of treatment may be required for noticeable results to develop. High molecular weight aliphatic alcohols appear to be safe at recommended doses. In the published clinical studies, only mild, short-term side effects such as nervousness, headache, diarrhea, and insomnia were seen. High molecular weight aliphatic alcohols appear to enhance the blood-thinning effects of aspirin, suggesting that unsupervised combination therapy could be dangerous. By the same principle, high molecular weight aliphatic alcohols should not be combined with other blood-thinning drugs, such as warfarin, heparin or pentoxifylline. There is also a chance that they might cause excessive bleeding if combined inappropriately with natural supplements that reduce clotting time, such as garlic, ginkgo and high doses of Vitamin E.

Vitamin D, for example in its common form, Vitamin D₃, is a compound with multiple activities. Its major function, and the one first ascribed to this vitamin is its requirement in mineralization and metabolism of bone. However more recent work has revealed an immune modulating effect of Vitamin D. It has been demonstrated, in the EAE mouse model of multiple sclerosis, to attenuate the inflammatory activity and associated tissue destruction.

Recent work has demonstrated that exogenous 1,25-dihydroxyvitamin D₃ can prevent experimental autoimmune encephalomyelitis, a widely accepted mouse model of human multiple sclerosis. The implication is that sufficient quantities of this vitamin may prevent the development of multiple sclerosis in genetically susceptible individuals. Multiple sclerosis patients often have a Vitamin D deficiency and commonly develop bone fractures. Furthermore, the vitamin may help stop the progression of the disease in those who suffer with it. The mechanism involved in this inhibition of disease progression may be related to the presence of Vitamin D receptors on activated lymphocytes of the immune system. As multiple sclerosis is considered an autoimmune disease, this finding may indicate an inhibitory role for the vitamin on lymphocyte activity, preventing subsequent tissue damage from immune reactions. In fact support for this concept is experimental work demonstrating the ability of Vitamin D to retard T-cell mediated immunity. The vitamin is an immunoregulatory hormone, augmenting the antiinflammatory helper T cells, T_{H}2, and suppressing the inflammatory, T_{H}1, cells, causing an increase in cytokines, IL-4, IL-5, IL-10, for the former, T_{H}2, and IFN-α, TNF-γ and IL-12 for the latter, T_{H}1.

Furthermore, Vitamin D also has been demonstrated to inhibit the progression of arthritis in mouse models of human rheumatoid arthritis. This type of arthritis is also considered to be a disease of autoimmunity, where the patient's immune system recognizes self as a foreign substance. Further, Vitamin D has been shown as effective in suppressing lesions in an animal model of psoriasis, another disease believed to contain an autoimmune component, as well as Type I diabetes, also referred to as insulin dependent diabetes myelitis (IDDM).

The intensity of the inflammatory response has been demonstrated to be influenced by the fatty acid constituents of cell membranes. The amount and type of fatty acid that becomes incorporated into cell membranes largely reflects the diet of the individual. Two polyunsaturated fatty acids, known as essential fatty acids and therefore which must be obtained from the diet, are the omega-6 and omega-3 fatty acids. These two fatty acids are key precursors to a class of bioactive lipids know as prostaglandins. The omega-6 fatty acid is present in the western diet in significant amounts, whereas the omega-3 fatty acids are present at very low levels in our diet today due to new agricultural and refinery techniques developed over the last 100 years, which deplete food of this fatty acid. A preponderance of the omega-6 fatty acids promotes the production of the pro-inflammatory 2-series of fat-derived hormone substances, the prostaglandins, whereas the omega-3 fatty acid promotes the anti-inflammatory 3-series of prostaglandins. The majority of experts agree that the ratio of these two fatty acids in our diet should be in the range of 1:1 to 1:2, omega-3 to omega-6. There is significant evidence to indicate that this ratio is more like 1:20 to 1:30 in the typical diet by people in the U.S., and that this unfavorable ratio may be contributing to diseases of inflammation including autoimmune disease. We simply ingest far too much omega-6 fat.

Coenzyme Q10 is a vitamin-like substance that functions as a carrier for the transport of electrons in the mitochondria during energy production. The molecule is critical for the health of the cell and low levels of it can impair cellular activity. The cholesterol-synthesis inhibiting drugs, such as the statins and policosanol, inhibit the synthesis of CoQ10 as it, like cholesterol, consists of the same building blocks, isoprene subunits.

Vitamin B₁₂, is necessary for several biochemical reactions in the body, many of which involve the transfer of methyl group. One of the relevant activities that requires Vitamin B₁₂ is the synthesis of myelin, the component surrounding and insulating the nerve and damaged during the course of the disease. Therefore, a deficiency in this vitamin can aggravate the symptoms of multiple sclerosis.

### SUMMARY

Provided are compositions for treating autoimmune disease, such as multiple sclerosis and rheumatoid arthritis, and inflammation, that appear to be substantially free of detrimental or toxic side effects in humans. In one most promising embodiment, the agent is a mixture of a long chain aliphatic primary alcohol in combination with one or more of a Vitamin D, a Vitamin B₁₂, a coenzyme Q and an omega-3 fatty acid. These compounds inhibit cholesterol synthesis, and attenuate the symptoms and risk factors common to autoimmune disease. Unlike the statin drugs, they are largely free of toxic side effects in humans. Though one or more of the compounds used to formulate the compositions described herein have been shown to demonstrate immune-modulating effects, the combinations described herein have demonstrated surprising synergy and effectiveness in treating autoimmune disease.

One embodiment provides compositions that attenuate multiple autoimmune disease risk factors. The compositions comprise as a first component, a high molecular weight aliphatic primary alcohol, and as a second component, one or more of a Vitamin D, a Vitamin B₁₂, a coenzyme Q (CoQ) and an omega-3 fatty acid. The long chain aliphatic primary alcohol can be 1-octacosanol, 1-triacontanol, 1-hexacosanol, 1-tetracosanol and/or 1-heptacosanol. The second component can be Vitamin D₃, Vitamin B₁₂, Coenzyme Q10 (CoQ10), docosahexaenoic acid (DHA) arid eicosapentaenoic acid (EPA).

Also provided are methods for attenuating one or more symptoms or risk factors of an autoimmune disease, or reducing one or more factors contributing to inflammation in a mammal, including the step of administering to the mammal an effective amount of a composition including a high molecular weight aliphatic primary alcohol and a second component selected from one or more of a Vitamin D, a Vitamin B₁₂, Coenzyme Q10 (CoQ10) and an omega-3 fatty acid, such as Vitamin D₃, Vitamin B₁₂, CoQ10, DHA and EPA. The methods provide for the administration to be continued until symptoms common to the autoimmune disease or inflammation have subsided. On return of the mammal to near normal physical state, the dosage may be reduced to a lower, maintenance-dose level. The methods typically contemplate prevention of tissue destruction associated with inflammation, and autoimmune diseases.

### DETAILED DESCRIPTION

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Provided are agents for treating autoimmune disease, such as multiple sclerosis and rheumatoid arthritis, and inflammation, that are virtually free of side effects in humans. In one most promising embodiment, the agent is a long chain aliphatic primary alcohol, or a mixture of long chain aliphatic primary alcohols. Compositions are provided that attenuate multiple autoimmune disease and inflammation risk factors. The compositions include as a first component, a high molecular weight aliphatic primary alcohol, and as a second component, one or more of a Vitamin D, a Vitamin B₁₂, Coenzyme Q10 (CoQ10) and an omega-3 fatty acid.

Preferred embodiments are directed towards compositions and methods of use thereof for coordinate reduction of multiple risk factors relating to autoimmune disease. Certain autoimmune disease risk factors are elevated levels of inflammatory agents, The composition includes, as a first component, at least one of a high molecular weight primary aliphatic alcohol, and as a second component at least one of Vitamin D₃, Vitamin B₁₂, CoQ10, DHA and EPA. The composition is useful in controlling symptoms associated with autoimmune disease and inflammation, whether or not associated with an autoimmune disease. In the Examples below, the composition is shown to be particularly useful in attenuating symptoms and risk factors associated with multiple sclerosis and rheumatoid arthritis, thereby “treating” these diseases.

The methods described herein provide therapeutic treatment and prevention of multiple risk factors related to autoimmune disease. Certain autoimmune disease risk: factors include elevated levels of TNF-α and IFN-γ. It appears that an imbalance in the ratio of two additional cytokines, IL-12 and IL-10 in these patients may trigger the production of TNF-α and IFN-γ. Furthermore, there is an imbalance in the ratio of the thymocyte-derived immune cells, T_{H}1 and T_{H}2 with T_{H}1 cells significantly elevated in number in autoimmune disease. Another risk factor, Rho, is a GTP binding protein, and recent information indicates that attenuation of tissue levels of this protein as well as related proteins, stimulates the synthesis of myelin and neurite growth. Furthermore, inhibition of Rho has been demonstrated to inhibit the migration (extravasation) of inflammatory white blood cells (lymphocytes) from the vasculature into the central nervous system. The methods provide for the administration of the composition to be continued until serum levels of these substances return to normal and or the symptoms of the disease, autoimmune disease, disappear or are attenuated to near normal.

As used herein, the term “dietary supplement” refers to compositions consumed to affect structural or functional changes in physiology. The term “therapeutic composition” refers to any compounds or combinations of compounds administered to treat or prevent a disease, that is, to prevent or attenuate a symptom or risk factor associated with the disease.

As used herein, the term “autoimmune disease” refers to a disease of the tissues of the body caused by immune-responsiveness against self-tissues and associated with production of inflammatory factors, which further promote tissue destruction. Autoimmune diseases either are systemic or organ-specific. Examples of systemic autoimmune diseases include: multiple sclerosis, rheumatoid arthritis, systemic lupus erythematosis, ankylosing spondylitis, scleroderna and Sjogren's syndrome. Examples or organ-specific autoimmune diseases include: Addison's disease, Autoimmune hemolytic anemia, Goodpasture's syndromne, Grave's disease, Hashimoto's thyroiditis, idiopathic thrombocytopenia purpura, insulin-dependent diabetes mellitus, myasthenia gravis, pernicious anemia, poststreptococcal glomerulonephritis and psoriasis. Myocardial infarction and spontaneous infertility also are known to have an autoimmune component in many instances.

As used herein, the term “inflammation” refers to a condition that results from the activation of cells of the immune defense system and their movement (extravasation) from vessels, such as lymph and blood vessels, into surrounding tissues of the body, to destroy what the cells believe to be invasion by a foreign substance. The consequence of the immune cell activation process is the subsequent release by these specialized immune cells of toxic substances and specific factors or cytokines purportedly to kill a pathogen. The cytokines and toxic substances cause an increase in vascular permeability and subsequent release of fluid into the area causing swelling, redness, and soreness. In autoimmune disease this process is thought to occur in response to an antibody reacting with the body's own antigens (self) instead of a true foreign invader. The result however is clear and that is the destruction of surrounding tissue.

As used herein, the term “cytokine” refers to a diverse group of cell-secreted low molecular weight proteins and peptides that regulate the intensity and duration of the immune response by exerting a variety of effects on lymphocytes and other immune cells. Cytokines typically act at nanomolar to picomolar concentrations under both normal and pathological conditions to modulate the functional activities of individual cells and tissues. Cytokines also can mediate interactions between cells directly and regulate processes taking place in the extra-cellular environment.

The term “Type-1 cytokine” refers to cytokines produced by T_{H}1 T-helper cells, responsible for classical cell-mediated immune responses. “Type-2 cytokines” are those produced by T_{H}2 T-helper cells, responsible for B-cell activation as part of a humoral immune response. Type-1 cytokines include IL-2, IFN-γ, IL-12 and TNF-α, while Type-2 cytokines include IL-4, IL-5 and IL-10.

As used herein, the term “fatty acid” refers to long chain aliphatic compounds beginning with a carboxylic acid moiety. The fatty acid eicosapentaenoid acid (EPA) is an omega-3 fatty acid with 5 unsaturated bonds, the first starting 3 carbons in from the last carbon (characteristic of omega-3 fatty acids) and alternating every third carbon toward the first carbon or carboxylic acid end. The fatty acid docosahexaenoic acid (DHA) is an omega-3 fatty acid with 6 unsaturated bonds.

As used herein, the term “policosanol” refers to mixtures of high molecular weight aliphatic alcohols ranging from 20 to 40 carbons in length. Some of the typical components are 1-octacosanol, 1-hexacosanol, 1-triacontanol, and 1-dotriacontanol. Policosanol can be isolated from a number of different natural sources, including sugar cane wax, rice bran wax, and beeswax. The preferred high molecular weight aliphatic alcohols of pharmaceutical grade, which can be obtained commercially, preferably pass extensive safety and efficacy procedures. An exemplary high molecular weight aliphatic alcohol product, known as “Rice Bran Wax”, is manufactured by Traco Labs, Inc. (Table 1). Non-limiting examples of policosanols and compositions including policosanol are provided in U.S. Pat. Nos. 5,856,316 and 6,355,274.

TABLE 1
composition of aliphatic alcohols in Rice Bran Wax (Traco Labs)

| | Approximate Percentage |
|---|---|
| | by weight of |
| Alcohol | Total Aliphatic Alcohol |
| 1-C₂₂OH (1-Docosanol) | 1.3 |
| 1-C₂₄OH (1-Tetracosanol) | 11.5 |
| 1-C₂₆OH (1-Hexacosanol) | 10.5 |
| 1-C₂₈OH (1-Octacosanol) | 20.1 |
| 1-C₃₀OH (1-Triacontanol) | 30.0 |
| 1-C₃₂OH (1-Dotriacontanol) | 16.7 |
| 1-C₃₄OH (1-Tetratriacontanol) | 8.0 |
| 1-C₃₆OH (1-Hexatriacontanol) | 1.8 |

Another commercial source of high molecular weight aliphatic alcohols (policosanols) is Garuda International (Lemon Cove, Calif.). This company supplies several products comprising high molecular weight aliphatic alcohols. One of these products is sold under the trademark LESSTANOL and comprises five aliphatic alcohols ranging from about 26 to 32 carbons in length (Table 2). An additional product from Garuda is OCTA-95, also isolated from sugar cane and comprises approximately 95% by weight (% wt.) 1-octacosanol.

TABLE 2
Composition of Garuda Sugar Cane Wax Extract (LESSTANOL)

| | Approximate % wt. |
|---|---|
| | of Total Aliphatic |
| Alcohol | Alcohol |
| C₂₄OH (1-Tetracosanol) | 0-10 |
| C₂₆OH (1-Hexacosanol) | 2 to 15 |
| C₂₇OH (1-Heptacosanol) | <0.5 |
| C₂₈OH (1-Octacosanol) | 55 to 70 |
| C₃₀OH (1-Triacontanol) | 5 to 20 |

As used herein, the term “high molecular weight aliphatic alcohols” are aliphatic alcohols, and “high molecular weight aliphatic primary alcohols∞ are 1-aliphatic alcohols having the formula CₙH₍₂ₙ₊₁)OH and mixtures thereof, wherein n is an integer having a value of about 20 to 40, and preferably from 22 to 40. High molecular weight aliphatic alcohols are components of policosanols. High molecular weight aliphatic alcohols typically are derived from plants, plant extracts, bees' wax or other natural sources.

Natural or synthetic metabolic intermediates, analogs or chemically modified forms of any referenced compound described herein are referred to as “derivatives” of those referenced compounds. Derivatives of any referenced compound have the same immunomodulatory action as the referenced compound although the relative immunomodulatory activity (bioactivity) of the derivative as compared to the referenced compound may vary, requiring different doses and dosage regimens, but with the same or substantially equivalent result. Derivatives of any referenced compound, unless expressly stated otherwise are considered to be a subset of the referenced compound. Therefore, “a D Vitamin” includes as a class Vitamin D derivatives and “a B₁₂ Vitamin” includes as a class Vitamin B₁₂ derivatives. Determining optimal doses and dosage regimens for any given compound can readily be determined by titration in experimental animals and in humans in a manner well known in the art. Derivatives may be modified in any fashion, with substitution of any group with another group that does not alter the biological function of that reference compound. Example of groups for chemical modifications include, without limitation: hydrogen; alkyl, alkoxyl, cycloalkyl, aryl, ester and ether groups, with or without hetero-atoms, including N, O and S; solubilizing groups, such as PEG groups; ionic groups and proteins or peptides.

As used herein, “coenzyme Q,” for example coenzyme Q10 or ubiquinone, is a specialized molecule largely residing in the mitochondrial compartment of the cell, and includes as a class any effective coenzyme Q and effective derivatives of coenzyme Q and coenzyme Q10. Coenzyme Q participates in oxidation-reduction reactions involving the transport of electrons during energy production in the mitochondria. Coenzyme Q is a quinone derivative with a long isoprenoid tail and the number of five-carbon isoprene units in coenzyme Q depends on the species. The most common form in mammals contains 10 isoprene units, hence the designation “coenzyme Q10.” CoQ10 is included in the compositions described herein, to help prevent policosanol-induced deficiency in this critical cellular compound. Additionally, CoQ10 is a potent antioxidant. This property is, an additional benefit as it can help attenuate the destructive effects on tissues from the well-known inflammation associated production of oxidants.

As used herein, a “D Vitamin” includes as a class cholecalciferol or Vitamin D₃ and derivatives thereof. A variety of D Vitamin derivatives are known in the art, for example, and without limitation, as are found in U.S. Pat. Nos. 6,537,980, 6,548,715, 6,573,255 and 6,613,920, which are incorporated herein by reference for their disclosure of useful D Vitamin derivatives.

As used herein, “a B₁₂ Vitamin” is a member of a class of compositions that includes cyanocobalamin (Vitamin B₁₂), methylcobalamin, adenosylcobalamin and all naturally occurring or synthetic metabolic intermediates and chemically altered variations of these compounds, referred to herein as B₁₂ Vitamin derivatives, that can perform their metabolic functions relating to methyl group transfer.

The compositions described herein include, generally, one or more high molecular weight aliphatic primary alcohols in combination with one of a Vitamin D, an omega-3 fatty acid, a coenzyme Q, a Vitamin B₁₂, and, optionally, an antioxidant, such as uric acid, alpha lipoic acid or natural vitamin E. In one embodiment, the composition includes policosanol, Vitamin D₃, omega-3 fatty acids, coenzyme Q10, Vitamin B₁₂ and an antioxidant. In certain embodiments, the composition contains one of both of the omega-3 fatty acids DHA and EPA. Examples of suitable high molecular weight aliphatic alcohol compounds, Vitamin D, Vitamin B₁₂, CoQ, and omega-3 fatty acids are listed in Table 3, those containing one asterisk (*) may be preferred and those containing two asterisks (**) may be particularly preferred.

| Class | Compound |
|---|---|
| High molecular | 1-C₂₂OH |
| weight aliphatic | |
| alcohols | |
| | 1-C₂₄OH* |
| | 1-C₂₆OH* |
| | 1-C₂₇OH* |
| | 1-C₂₈OH** |
| | 1-C₃₀OH** |
| | 1-C₃₂OH |
| | 1-C₃₄OH |
| | 1-C₃₆OH |
| | 1-C₃₈OH |
| | 1-C₄₀OH |
| B₁₂ Vitamin | cyanocobalamin* |
| | methylcobalamin** |
| | adenosylcobalamin* |
| D Vitamin | Vitamin D₃** |
| | Vitamin D₂* |
| | 25-OH-Vitamin D₃* |
| | 24,25-OH-Vitamin D₃* |
| | 1,25-OH-Vitamin D₃* |
| Coenzyme Q | coenzyme Q10** |
| Omega-3 fatty acids | DHA** |
| | EPA* |

The composition contains, for example and without limitation, permutations of the compounds presented in Table 3, including one or more high molecular weight alcohols and, optionally, one of the listed B₁₂ Vitamin, D Vitamin, coenzyme Q and omega-3 fatty acid compounds. One embodiment is a composition including at least one of 1-octacosanol, 1-triacontanol, 1-tetracosanol, 1-heptacosanol, 1-hexacosanol, and at least one of Vitamin D₃, Vitamin B₁₂, Coenzyme Q10, and an omega-3 fatty acid.

By attenuating multiple autoimmune risk factors, such as reducing levels of T_{H}1-derived cytokines and raising T_{H}2-derived cytokines, increasing serum levels of immunomodulating Vitamin D, as well as increasing serum levels of the methyl donor, Vitamin B₁₂, and coenzyme Q10, which may be decreased in amounts in the mammal taking cholesterol-lowering compounds such as high molecular weight aliphatic-primary alcohols, the present compositions and methods therefore significantly reduce (attenuate) the symptoms and risks of autoimmune disease.

In embodiments containing coenzyme Q10, Vitamin D₃, Vitamin B₁₂ and/or omega-3 fatty acids, the weight ratio of high molecular weight aliphatic alcohols to coenzyme Q10 is about 1:1 to 1:1000. The weight ratio of high molecular weight aliphatic alcohols to Vitamin D₃ is about 1:0.0001 to 1:1. The weight ratio of high molecular weight aliphatic alcohols to Vitamin B₁₂ is about 1:0.001 to 1:1. The weight ratio of high molecular weight aliphatic alcohols to omega-3 fatty acids is about 1:0.1 to 1:10.

In one embodiment, the composition is formulated to deliver from about 0.1 mg to about 1000 mg of one or more high molecular weight aliphatic alcohol and from about 1.2 mg to about 1200 mg CoQ10. In another embodiment, the composition is formulated to deliver from about 1 mg to about 100 mg of one or more high molecular weight aliphatic alcohol and from about 1 μg to about 100 μg of Vitamin D₃.

In further embodiments, the composition is formulated to deliver from about 0.1 mg to about 100 mg of one or more high molecular weight aliphatic alcohol in combination with from about 1.2 mg to about 1200 mg of CoQ10; from about 1 μg to about 100 μg Vitamin D₃; from about 0.1 mg to about 10 mg or from about 1 μg to about 5 mg Vitamin B₁₂, and/or about 1 g to about 10 g of one or more omega-3 fatty acid. In another embodiment, the composition is formulated to deliver about 0.2 mg to about 50 mg of one or more high molecular aliphatic alcohol in combination with from about 2.5 mg to about 600 mg of CoQ10; from about 2 μg to about 50 μg of Vitamin D₃; from about 2 μg to about 2.5 mg of Vitamin B₁₂ and/or from about 0.25 g to about 40 g or from about 1 g to about 5 g of one or more omega-3 fatty acid.

In addition to the active ingredients described, the composition can include various additives such as other natural components of intermediary metabolism, vitamins, minerals, and natural plant products. Examples of such natural products include green tea, white tea, black tea, stinging nettle, milk thistle, gingko, curcumin, grape seed extract, resveratrol, creatine, and lycopene. Examples of some components of intermediary metabolism include alpha lipoic acid, acetyl-L-carnitine, L-taurine, and L-arginine. Examples of vitamins include, folic acid, ascorbic acid, biotin, thiamine, pantothenic acid, pyridoxal phosphate (B₆), and d-α-tocopherol (natural Vitamin E). Examples of minerals include; calcium, selenium, zinc, and magnesium. Other inert ingredients, referred to herein as “pharmaceutically acceptable carriers” include, without limitation: fillers, solvents, dispersion media, isotonic and absorption-delaying agents, diluents, binders, adhesives, bulking agents, viscosity modifiers, lubricants, coatings, colorings, flavorings, fragrances, sweeteners, fats, oils, food substances, buffers, amino acids, amino sugars, oligosaccharides and polysaccharides, absorbents and solubilizing agent, such as magnesium stearate, hydroxypropyl methylcellulose, titanium dioxide. The use and methods of use of such pharmaceutically acceptable carriers for pharmaceutical, nutraceutical and dietary supplement formulations and products is well known in the art. Except insofar as any such pharmaceutically acceptable carrier is incompatible with the active ingredients, its use in the present composition is contemplated. In one embodiment, talc and magnesium stearate, are included in the formulation. Other pharmaceutically acceptable carriers may be used in the manufacture of the composition as a dietary bar, liquid, tablet, capsule or functional food, can include flavorings, sugars, aminosugars, proteins and/or modified starches, as well as limited fats and oils.

The dietary supplement or therapeutic composition can be formulated in any manner known by one of skill in the art. The composition may be formulated into a solid or particle-filled capsule, caplet, tablet, softgel gelatin cube, suppository, transdermal patch, systemic implant, liquid, food bar, functional food, or an injectable or oral solution of suspension, using techniques available to one of skill in the art. However, provided the proper daily dosage is incorporated, the present compositions may also be formulated in other convenient forms, such as a solution or suspension, a spray solution or suspension, a liquid, a food, or snack item. Food, snack, or liquid items can include any ingestible ingredients, including sweeteners, flavorings, oils, starches, proteins, fruits or fruit extracts, vegetables, or vegetable extracts, grains, animal fats or proteins. Thus, the compositions can be formulated into cereals, snack items such as chips, bars, gumdrops, or chewable candies.

Also provided are methods of reducing risk factors and symptoms of autoimmune disease and/or inflammation in mammals and for prophylactic treatment of autoimmune disease and/or inflammation. The method includes administering to the animal a composition described or set forth herein for a period of time and in an amount effective to reduce symptoms of the autoimmune disease, such as, in the case of multiple sclerosis, and without limitation, numbness, pain, swelling, optic neuritis, and symptoms arising from inflammatory reactions. Symptoms of each of the various autoimmune diseases are known to the medical sciences. Methods of reducing serum markers of inflammation, such as cytokines and inflammatory cells, also are provided. The methods of reducing risk factors and symptoms, and for prophylactically preventing such risk factors and/or symptoms provide effective treatment of the autoimmune disease and/or inflammation.

Since many modifications, variations and changes in detail can be made to the described preferred embodiments, it is intended that all matters in the foregoing description and the following examples are interpreted to illustrate and not in any way to be limiting.

### EXAMPLE 1

### Tablet Formulation Containing High Molecular Aliphatic Alcohols, Vitamin D₃, Omega-3 Fatty Acids, Coenzyme Q10 and Vitamin B₁₂

The formulation listed below in Table 4 would be administered once or twice per day for treatment of autoimmune disease and/or inflammation, namely to attenuate risk factors and symptoms associated with the autoimmune disease and/or inflammation.

TABLE 4
Tablet Formulation

| | Weight (% weight |
|---|---|
| | active ingredients) |
| Ingredient | (approximate) |
| High molecular weight aliphatic primary | 40 mg (1.9%) |
| alcohols | |
| Vitamin D₃ | 0.050 mg (0.0023%) |
| Omega-3 fatty acids (DHA:EPA 1:1) | 2000 mg (93.3%) |
| Coenzyme Q10 | 100 mg (4.7%) |
| Vitamin B₁₂ (methylcyanocobalamin) | 2 mg (0.09%) |

### EXAMPLE 2

### Formulation Containing High Molecular Weight Aliphatic Primary Alcohols, Vitamin D₃, Omega-3 Fatty Acids, Coenzyme Q10, and Vitamin B₁₂

The formulation listed in Table 5 would be administered once or twice per day for treatment of autoimmune disease and/or inflammation, namely to attenuate risk factors and symptoms associated with the autoimmune disease and/or inflammation.

TABLE 5
Formulation

| | Weight (% weight |
|---|---|
| | active ingredients) |
| Ingredient | (approximate) |
| High molecular weight aliphatic primary | 40 mg (3.5%) |
| alcohols | |
| Vitamin D₃ | 0.050 mg (0.0044%) |
| Omega-3 Fatty Acids (DHA:EPA 1:1) | 1000 mg (87.6%) |
| Coenzyme Q10 | 100 mg (8.8%) |
| Vitamin B₁₂ (methylcyanocobalamin) | 2 mg (0.175%) |

### EXAMPLE 3

### Formulation Containing High Molecular Weight Aliphatic Primary Alcohols, Vitamin D₃, Omega-3 Fatty Acids, Coenzyme Q10, and Vitamin B₁₂

The formulation listed in Table 6 would be administered once or twice per day for treatment of autoimmune disease and/or inflammation, namely to attenuate risk factors and symptoms associated with the autoimmune disease and/or inflammation.

TABLE 6
Formulation

| | |
|---|---|
| | Weight (% weight |
| | active ingredients) |
| Ingredient | (approximate) |

| | |
|---|---|
| High molecular weight aliphatic primary | 10 mg (1.3%) |
| alcohols | |
| Vitamin D₃ | 0.0125 mg (0.0016%) |
| Omega-3 fatty acids (DHA:EPA 1:1) | 750 mg (95%) |
| Coenzyme Q10 | 25 mg (3.2%) |
| Vitamin B₁₂ (methylcyanocobalamin) | 1 mg (0.13%) |

### EXAMPLE 4

### Formulation Containing High Molecular Weight Primary Aliphatic Alcohols, Vitamin D₃, Omega-3 Fatty Acids, Coenzyme Q10 and Vitamin B₁₂

The formulation listed in Table 7 would be administered once or twice per day for treatment of autoimmune disease and/or inflammation, namely to attenuate risk factors and symptoms associated with the autoimmune disease and/or inflammation.

TABLE 7
Formulation

| | Weight (% weight |
|---|---|
| | active ingredients) |
| Ingredient | (approximate) |
| High molecular weight aliphatic primary | 10 mg (0.123%) |
| alcohols | |
| Vitamin D₃ | 0.025 mg (0.0031%) |
| Omega-3 fatty acids (DHA:EPA 1:1) | 750 mg (92%) |
| Coenzyme Q10 | 50 mg (6.1%) |
| Vitamin B₁₂ (methylcyanocobalamin) | 1 mg (0.12%) |

### EXAMPLE 5

### Attenuation of Risk Factors in Patients with Multiple Sclerosis by Formulations Containing High Molecular Weight Alcohols

Two patients, both females ages 38 (patient A) and 56 (patient B) with histories of multiple sclerosis, both being diagnosed with the disorder at age 28, were enrolled in the study. Patient A was experiencing an acute case of optic neuritis, a common symptom of multiple sclerosis, and patient B was experiencing numbness throughout her body, more pronounced on her left side, as well as a tingling sensation in her hands and feet. In addition, patient B had significant problems with coordination, which were exhibited in the form of tripping and irregular gait. Both patients A and B were easily tired, energy deficient, and rarely felt rested even after 8-9 hours of sleep/night.

A third and very recent patient to participate in this study, patient C, is 50 years old and first exhibited symptoms of MS at the age of 27. She has numbness and tingling in her arms and legs and requires the aid of a walker to get around.

Patients A, B and C received the formulation described in Table 7. The source of the policosanols used in the formulation is Garuda International. A profile of the aliphatic alcohol composition in Table 7 is presented in Table 2. Patient A was instructed to take two tablets twice daily, and Patients B and C were instructed to take one tablet twice daily with breakfast and evening meals, until the symptoms totally, or nearly totally disappeared. On disappearance of the symptoms, Patient A was instructed to lower the dose to one half, or 1 tablet twice daily, whereas Patients B and C were instructed to continue with the dose of one tablet-twice daily, with breakfast and evening meals.

The results were dramatic in Patients A and B. Patient A experienced a gradual but steady decrease in intensity of symptoms of optic neuritis, blurry vision, pain in eye and tired-weak physical state, starting with day two of treatment with the formulation. By day 14 she reported complete remission of all symptoms and started on the maintenance dose of two tablets/day. Patient B experienced a gradual but steady return of feeling to her left side and midriff starting with day 5 and by day 17 had reported a total remission of all symptoms including tingling sensation, coordination, tripping; she continued with her dose of two tablets/day. Both Patients A and B have continued to improve with respect to energy level and are less tired during the day than before the treatment commenced.

Patient B experienced for the first time in over 10 years complete disappearance of one or more of the symptoms she was experiencing before the start of the treatment. These symptoms include tingling sensation in hands and feet, problems with walking with episodes of frequent tripping, midriff and left side numbness, hand tremor and low energy state. Even more impressive is that her physical condition continues to improve with time. She has now been on the composition (two tablets/day) for almost 10 months. Her sense of balance has improved significantly. This is evident from her reported ability to use and climb a stepladder for the first time in over ten years. She also describes a significant improvement in the stability of her hands (hand tremor); she mentions being able to carry out activities with her hands, activities that previously were nearly impossible due to severe hand tremor.

Patient A has not had a single relapse (optic neuritis) since starting on the composition over nine months ago. She reports feeling excellent with an energy level nearly equal to that before her diagnosis With MS. She continues to take the maintenance dose of two tablets/day.

Patient C has only recently started taking the composition and has been on it for about two weeks. Patient C has a severe case of MS and can only walk with the aid of a walker. She has commented that although her legs are weak, she has noticed significant improvement in her ability to get around with the aid of her walker since starting on the composition.

### EXAMPLE 6

### Attenuation of Risk Factors in Patients with Rheumatoid Arthritis with Formulations Containing High Molecular Weight Alcohols

One patient, Patient D, is a 59 year old female who was diagnosed with severe rheumatoid arthritis in 1994, nine years ago. Patient D started on the formulation (as described in Table 7) as for Patients A, B and C. She was instructed to take four tablets/day and to continue the formulation until her symptoms subsided. Patient D reported a noticeable improvement in her condition within 2-3 weeks. Improvements included less pain in her joints, more energy and an overall improvement in her quality of life. She currently alternates between taking two and four tablets/day depending on whether she feels her condition is stable or worsening. Unlike the MS patients, her condition appears to require a continued alternate high-low dose regimen on a more frequent basis. Patient D has been on the regimen now for over six months and is pleased with her progress since starting on the formulation.

1. A composition for attenuating at least one factor involved in the inflammation-associated destruction of tissue comprising a first component comprising a long-chain normal primary aliphatic alcohol, and a second component selected from the group consisting of a B₁₂ Vitamin, a D Vitamin, coenzyme Q, tan omega-3 fatty acid and combinations thereof.

2. The composition of claim 1, wherein the second component of the composition comprises an omega-3 fatty acid in combination with one or more of a B₁₂ Vitamin, a D Vitamin and coenzyme Q10.

3. The composition of claim 1, wherein the second component of the composition comprises a B₁₂ Vitamin, a D Vitamin, coenzyme Q10 and an omega-3 fatty acid.

4. The composition of claim 1, wherein the long-chain normal primary aliphatic alcohol comprises alcohols having the formula CₙH₍₂ₙ₊₁₎OH, and mixtures thereof, wherein n is an integer having a value from 22 to 40.

5. The composition of claim 1, wherein the long-chain normal primary aliphatic alcohol comprises one or more of 1-triacontanol, 1-octacosanol, 1-heptacosanol 1-hexacosanol, 1-tetracosanol.

6. The composition of claim 1, wherein the long-chain normal primary aliphatic alcohol having the formula C₂₈H₅₇OH comprises between 50% to 70% of the alcohols present.

7. The composition of claim 1, wherein the long-chain normal primary aliphatic alcohol having the formula C₃₀H₆₁OH comprises between 50% to 70% of the alcohols present.

8. The composition of claim 1, wherein the second component of the composition comprises, a B₁₂ Vitamin selected from the group consisting of cyanocobalamin, methylcobalamin, adenosylcobalamin and mixtures thereof

9. The composition of claim 8, wherein the B₁₂ Vitamin is methylcobalamin.

10. The composition of claim 1, wherein the second component of the composition comprises a D Vitamin selected from the group consisting of Vitamin D₂, Vitamin D₃, 1,25-dihydroxyvitamin D₃, 24,25-dihydroxyvitamin D₃, 25-hydroxyvitamin D₃ and mixtures thereof.

11. The composition of claim 10, wherein the D Vitamin is Vitamin D₃.

12. The composition of claim 1, wherein the second component of the composition comprises an omega-3 fatty acid selected from the group consisting of eicosapentaenoic acid, docosahexaenoic acid and mixtures thereof.

13. The composition of claim 1, wherein the composition contains the long-chain normal primary aliphatic alcohol in an amount between 0.1 mg to 1000 mg per dosage.

14. The composition of claim 1, wherein the composition contains the long-chain normal primary aliphatic alcohol in an amount between 0.1 mg to 100 mg per dosage.

15. The composition of claim 1, wherein the composition contains the long-chain normal primary aliphatic alcohol in an amount between 0.2 mg to 50 mg per dosage.

16. The composition of claim 1, wherein the second component of the composition contains the B₁₂ Vitamin in an amount of about 0.1 mg to 10 mg per dosage.

17. The composition of claim 1, wherein the second component of the composition contains the B₁₂ Vitamin in an amount of about 0.001 mg to 5 mg per dosage.

18. The composition of claim 1, wherein the second component of the composition contains the B₁₂ Vitamin in an amount of about 0.002 mg to 2.5 mg per dosage.

19. The composition of claim 1, wherein the second component of the composition contains the D Vitamin in an amount of about 0.001 mg to 0.100 mg per dosage.

20. The composition of claim 1, wherein the second component of the composition contains the D Vitamin in an amount of about 0.002 mg to 0.050 mg per dosage.

21. The composition of claim 1, wherein the second component of the composition contains the coenzyme Q10 in an amount between 1.2 mg and 1200 mg per dosage.

22. The composition of claim 1, wherein the second component of the composition contains the coenzyme Q10 in an amount between 2.5 mg and 600 mg per dosage.

23. The composition of claim 1, wherein the second component of the composition contains the omega-3 fatty acid in an amount between 0.25 g to 40 g per dosage.

24. The composition of claim 1, wherein the second component of the composition contains the omega-3 fatty acid in an amount between 1 g to 10 g per dosage.

25. The composition of claim 1, wherein the second component of the composition contains the omega-3 fatty acid in an amount between 1 g to 5 g per dosage.

26. The composition of claim 1, wherein the omega-3 fatty acid is comprised of a mixture of eicosapentaenoic acid and docosahexaenoic acid in a ratio of between 1:1 to 1:5.

27. The composition of claim 1, further comprising at least one member selected from the group consisting of antioxidants, Vitamins, minerals, proteins, fats, carbohydrates, natural plant products, and mixtures thereof.

28. The composition of claim 1, further comprising at least one pharmaceutically acceptable carrier, wherein the pharmaceutically acceptable carrier is selected from the group consisting of diluents, stabilizers, binders, buffers, lubricants, coating agents, preservatives, emulsifiers and suspension agents.

29. The composition of claim 1, wherein the composition is formulated as one of a capsule, a caplet, a tablet, a softgel, a gelatin cube, a suppository, a patch, a systemic implant, a liquid, a bar, a functional food, an oral solution, an oral suspension, an injectable solution and an injectable suspension.

30. A method for attenuating one or more symptoms or risk factors associated with autoimmune disease or immuno-inflammatory disease in mammals comprising administering an effective amount of a composition comprising a first component comprising a long-chain normal primary aliphatic alcohol, and a second component selected from the group consisting of a B₁₂ Vitamin, a D Vitamin, a coenzyme Q, an omega-3 fatty acid and combinations thereof.

31. The method of claim 30, wherein the second component of the composition comprises an omega-3 fatty acid in combination with one or more of a B₁₂ Vitamin, a D Vitamin, and a coenzyme Q, and combinations thereof.

32. The method of claim 30, wherein the second component of the composition comprises a B₁₂ Vitamin, a D Vitamin, coenzyme Q10, and an omega-3 fatty acid.

33. The method of claim 30, wherein the long-chain normal primary aliphatic alcohol comprises alcohols having the formula CₙH₍₂ₙ₊₁₎OH, and mixtures thereof, wherein n is an integer having a value from 22 to 40.

34. The method of claim 30, wherein the long-chain normal primary aliphatic alcohol comprises one or more of 1-triacontanol, 1-octacosanol, 1-heptacosanol, 1-hexacosanol, 1-tetracosanol.

35. The method of claim 30, wherein the long-chain normal primary aliphatic alcohol having the formula C₂₈H₅₇OH comprises between 50% to 70% of the alcohols present.

36. The method of claim 30, wherein the long-chain normal primary aliphatic alcohol having the formula C₃₀H₆OH comprises between 50% to 70% of the alcohols present.

37. The method of claim 30, wherein the second component of the composition comprises a B₁₂ Vitamin selected from the group consisting of cyanocobalamin, methylcobalamin, adenosylcobalamin and mixtures thereof.

38. The method of claim 37, wherein the B₁₂ Vitamin is methylcobalamin.

39. The method of claim 30, wherein the second component of the compound comprises a D Vitamin selected from the group consisting of Vitamin D₂, Vitamin D₃, 1,25-dihydroxyvitamin D₃, 24,25-dihydroxyvitamin D₃, 25-hydroxyvitamin D₃ and mixtures thereof.

40. The method of claim 39, wherein the D Vitamin is Vitamin D₃.

41. The method of claim 30, wherein the second component of the compound comprises an omega-3 fatty acid selected from the group consisting of eicosapentaenoic acid, docosahexaenoic acid and mixtures thereof.

42. The method of claim 30, wherein the composition contains the long-chain normal primary aliphatic alcohol in an amount between 0.1 mg to 1000 mg per dosage.

43. The method of claim 30, wherein the composition contains the long-chain normal primary aliphatic alcohol in an amount between 0.1 mg to 100 mg per dosage.

44. The method of claim 30, wherein the composition contains the long-chain normal primary aliphatic alcohol in an amount between 0.2 mg to 50 mg per dosage.

45. The method of claim 30, wherein the second component of the composition contains the B₁₂ Vitamin in an amount of about 0.1 mg to 10 mg per dosage.

46. The method of claim 30, wherein the second component of the composition contains the B₁₂ Vitamin in an amount of about 0.002 mg to 5 mg per dosage.

47. The method of claim 30, wherein the second component of the composition contains the B₁₂ Vitamin in an amount of about 0.002 mg to 2.5 mg per dosage.

48. The method of claim 30, wherein the second component of the composition contains the D Vitamin in an amount of about 0.001 mg to 0.100 mg per dosage.

49. The method of claim 30, wherein the second component of the composition contains the D Vitamin in an amount of about 0.002 mg to 0.050 mg per dosage.

50. The method of claim 30, wherein the second component of the composition contains the coenzyme Q10 in an amount between 1.2 mg and 1200 mg per dosage.

51. The method of claim 30, wherein the second component of the composition contains the coenzyme Q10 in an amount between 2.5 mg and 600 mg per dosage.

52. The method of claim 30, wherein the second component of the composition contains the omega-3 fatty acid in an amount between 0.25 g to 40 g per dosage.

53. The method of claim 30, wherein the second component of the composition contains the omega-3 fatty acid in an amount between 1 g to 10 g per dosage.

54. The method of claim 30, wherein the second component of the composition contains the omega-3 fatty acid in an amount between 1 g to 5 g per dosage.

55. The method of claim 30, wherein the second component of the composition comprises an omega-3 fatty acid comprised of a mixture of eicosapentaenoic acid and docosahexaenoic acid in a ratio of between 1:1 to 1:5.

56. The method of claim 30, wherein the composition further comprises at least one member selected from the group consisting of antioxidants, vitamins, minerals, proteins, fats, carbohydrates, natural plant products, and mixtures thereof.

57. The method of claim 30, wherein the composition further comprises at least one pharmaceutically acceptable carrier, comprising one of a diluent, a stabilizer, a binder, a buffer, a lubricant, a coating agent, a preservative, an emulsifier and a suspension agent.

58. The method of claim 30, wherein the composition is formulated as one of a solid capsule, a caplet, a tablet, a softgel, a gelatin cube, a suppository, a patch, a systemic implant, a liquid, a bar, a functional food, and an oral or injectable solution or suspension.

59. The method of claim 30, wherein the autoimmune disease or immuno-inflammatory disease is one of multiple sclerosis and rheumatoid arthritis.

60. The method of claim 30, wherein the autoimmune disease or immuno-inflammatory disease is multiple sclerosis.

61. The method of claim 30, wherein the autoimmune disease or immuno-inflammatory disease is rheumatoid arthritis.

62. The method of claim 30, wherein the composition is administered until the symptoms of the autoimmune disease or immuno-inflammatory disease are attenuated to a non-detectable level.

63. The method of claim 30, further comprising administering the composition at a reduced maintenance level after the symptoms of the autoimmune disease or immuno-inflammatory disease are attenuated to a near non-detectable level.

64. The method of claim 30, wherein the composition is administered prophylactically to prevent the medical condition of the autoimmune disease or immuno-inflammatory disease.
